(19) United States
(12) Patent Application Publication
(10) Pub. No.: US 2007/0198094 A1
(43) Pub. Date: Aug. 23, 2007

(54) Adaptor prosthesis kit

(75) Inventors: Brian K. Berelsman, Warsaw, IN (US); Nathan A. Winslow, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/357,794

(22) Filed: Feb. 17, 2006

(51) Int. Cl. A61F 2/40; A61F 2/36; A61F 2/28

(52) U.S. Cl. 623/19.14; 623/23.47; 623/22.42

(57) ABSTRACT

The present disclosure is directed to a kit of prosthetic components having at least two monolithic adaptors. The first adaptor incorporates a first and a second coupling mechanism, where the height of the first coupling mechanism defines the distance between an articulating surface and a fixture. The second monolithic adaptor also includes a third and a fourth coupling mechanism. The height of the third coupling mechanism can also define the distance between the articulating surface and the fixture. The coupling mechanisms, that define the distance between the articulating surface and the fixture, do not have equal heights.

### FIELD

The present disclosure relates to a kit of prosthetic components for repair and reconstruction of a portion of a modified ball-and-socket joint, and more specifically a kit of adaptors used to vary the distance between an articulating prosthetic surface and a bone fixation component.

### BACKGROUND

The shoulder joint is considered to be one of the most complex joints in the body. The scapula, the clavicle, and the humerus all meet at the shoulder joint. The head of the humerus fits into a shallow socket of the scapula called the glenoid fossa to form a mobile joint. When the joint is articulated, the humeral head moves in the glenoid fossa to provide a wide range of motion. The shoulder joint may suffer from various maladies including rheumatoid arthritis, osteoarthritis, rotator cuff arthropathy, vascular necrosis or bone fracture. If severe joint damage occurs and no other means of treatment is found to be effective, then a hemi or total shoulder reconstruction may be necessary.

A shoulder joint prosthesis generally includes the replacement of the ball of the humerus and, optionally, the socket of the shoulder blade with specially designed artificial components. The bio-kinematics, and thus the range of motion in the shoulder vary greatly among prospective patients for reconstructive shoulder surgery. The humeral component typically has a metal shaft or stem with a body portion that is embedded in the resected humerus and a generally hemispherical head portion supported on the stem. The head slidingly engages a glenoid implant on the glenoid fossa. During reconstructive surgery, the components of the prosthesis are matched with the bio-kinematics of the patient in an effort to maintain the natural range of motion of a healthy shoulder joint. Thus, a shoulder prosthesis design must be readily adaptable to a wide range of bio-kinematics for prospective patients.

In this regard, shoulder prostheses are generally available as either unitary structures or modular components. With an unitary shoulder prosthesis, a large inventory of differently sized components must be maintained to accommodate the different bone sizes and joint configurations of the prospective patients. With such an unitary shoulder prosthesis, the patient is typically evaluated by x-ray to determine the approximate component size needed for reconstruction. A number of differently sized components are selected as possible candidates based upon this preliminary evaluation. Final selection of the appropriately sized prosthesis is made during the surgery.

Modular prostheses systems which reduce the need to maintain large inventories of various sized components are well known in the art. Conventionally, the humeral prosthesis includes two components—a humeral stem component and a spherical head releasably coupled to the stem. Alternately, a three component design is known in which the stem and shoulder are interconnected with an adaptor. While providing an advantage over the unitary design in reducing the number of components needed, a rather large inventory of head components and/or adaptor components must be maintained to provide the desired range of geometric configurations with the conventional modular shoulder prostheses. Therefore, there is a need for modular shoulder prostheses which are readily adaptable to provide a range of geometric configurations, i.e. radial offsets, vertical offsets, and angular inclinations while minimizing the number of components required.

### SUMMARY OF THE INVENTION

A modular adaptor prosthesis kit is provided in accordance with the teachings of the present disclosure. The kit can include a set of adaptors for a shoulder or hip prosthesis, which cooperates with an articulating prosthetic head and fixation component, to provide a range of radial offsets, and/or vertical offsets, and/or angular inclinations. The adaptors are configured for use with a total shoulder or hip prosthesis.

A kit of prosthetic components is provided for adjustable vertical offset of a base of an articulating surface from the base of a fixation component. The kit can include a first monolithic adaptor having two coupling mechanisms. The height of the first coupling mechanism determines the vertical offset between the base of the articulating surface and the base of the fixation component. The kit can also include a second monolithic adaptor having at least two more coupling mechanisms. The height of one of the coupling mechanisms, of the second adaptor, also determines the vertical offset between the base of the articulating surface and the base of the coupling mechanism of the second adaptor. The height of the coupling mechanism of the second adaptor is not equal to the height of the first coupling mechanism.

Alternatively, a kit of prosthetic components can include a first prosthetic having an articulating surface and a fixation element having a coupling mechanism. A first monolithic adaptor implant is also provided and has a main body and a coupling mechanism. The main body and coupling mechanism define a first length between the articulating surface and the coupling mechanism. Similarly, a second monolithic adaptor implant is provided, also containing a main body and a coupling mechanism. The second length of the second adaptor defines the distance between the articulating surface and coupling mechanism and is not equal to the first length of the first adaptor.

The kit of prosthetic components can also incorporate at least two monolithic adaptors. The first adaptor has a main body and a coupling mechanism, where the adaptor is coupled to a member, having an articulating surface, and coupled to a fixation component. The distance between the articulating surface and the fixation component is defined by the height of the coupling mechanism. Likewise, a second monolithic adaptor is provided, which also has a main body and a coupling mechanism. The coupling mechanism on the second adaptor similarly defines the distance between the articulating surface and the fixation component. The height of the coupling mechanism on the second adaptor is not equal to the height of the coupling mechanism on the first adaptor.

The adaptor prosthesis kit of the present disclosure provides great flexibility in the adjustment of important bio-kinematic parameters for joint prosthesis systems, while minimizing the number of components required for the modular system. These and other features of the present disclosure will become apparent from the description and especially taken in conjunction with the accompanying exemplary drawings.

### BRIEF DESCRIPTION OF THE DRAWINGS

The various advantages of the present disclosure will become apparent to one skilled in the art upon reading the following specification and with reference to the drawings in which:

FIG. 1A is a side view of a joint prosthesis system incorporating an adaptor and spacer according to the present teachings;

FIG. 1B is a side view of a joint prosthesis system incorporating an adaptor prosthesis having an increased main body height;

FIG. 1C is a side view of a joint prosthesis system incorporating an adaptor prosthesis having an increased coupling mechanism height;

FIG. 1D is a side view of a joint prosthesis system incorporating an adaptor and offset plate;

FIG. 2A is a perspective view of an adaptor prosthesis;

FIG. 2B is a side view of the adaptor prosthesis shown in FIG. 2A;

FIG. 3A is a perspective view of an adaptor prosthesis incorporating a spacer;

FIG. 3B is an exploded perspective view of the adaptor prosthesis and spacer combination shown in FIG. 3A;

FIG. 3C is an exploded side view of the adaptor prosthesis and spacer combination shown in FIG. 3A;

FIG. 3D is a perspective view showing the intersection and vertical offset of the adaptor prosthesis and spacer of FIG. 3A;

FIG. 3E is a side view of the adaptor and spacer combination shown in FIG. 3A, further illustrating the spacer offset;

FIG. 4 is a side view of an adaptor prosthesis for use with the present disclosure;

FIG. 5 is a side view of an alternate adaptor prosthesis, having an increased main body height, for use with the present disclosure;

FIG. 6 is a side view of an alternate adaptor prosthesis, having an increased coupling mechanism height, for use with the present disclosure;

FIG. 7 is a side view of an alternate adaptor prosthesis, having an increased coupling mechanism width, for use with the present disclosure;

FIG. 8A is a perspective view of the adaptor prosthesis in FIG. 5;

FIG. 8B is a side view of the adaptor prosthesis of FIG. 5, illustrating the increased main body height;

FIG. 8C is a rotated perspective view of the adaptor prosthesis in FIG. 5, having an increased main body height;

FIG. 9A is a perspective view of the adaptor prosthesis depicted in FIG. 6;

FIG. 9B is a side view of the adaptor prosthesis of FIG. 6, illustrating an increased coupling mechanism height; and

FIG. 9C is a rotated perspective view of the adaptor prosthesis in FIG. 6, having an increased coupling mechanism height.

### DETAILED DESCRIPTION OF THE DISCLOSURE

The following description discloses a kit of modular adaptor components for use with a joint prosthesis system. The modular adaptors provide adjustment of the vertical offset between an articulating prosthetic head and fixation component, such as the humeral bone, and adjustment of the radial offset and/or angular inclination of the head relative to the stem. These descriptions are merely exemplary and are not intended to be limiting in application or use. Moreover, while the present disclosure is described in detail with respect to a shoulder joint or hip joint prosthesis system, it will be appreciated by those skilled in the art that the present disclosure is not limited to the details illustrated herein.

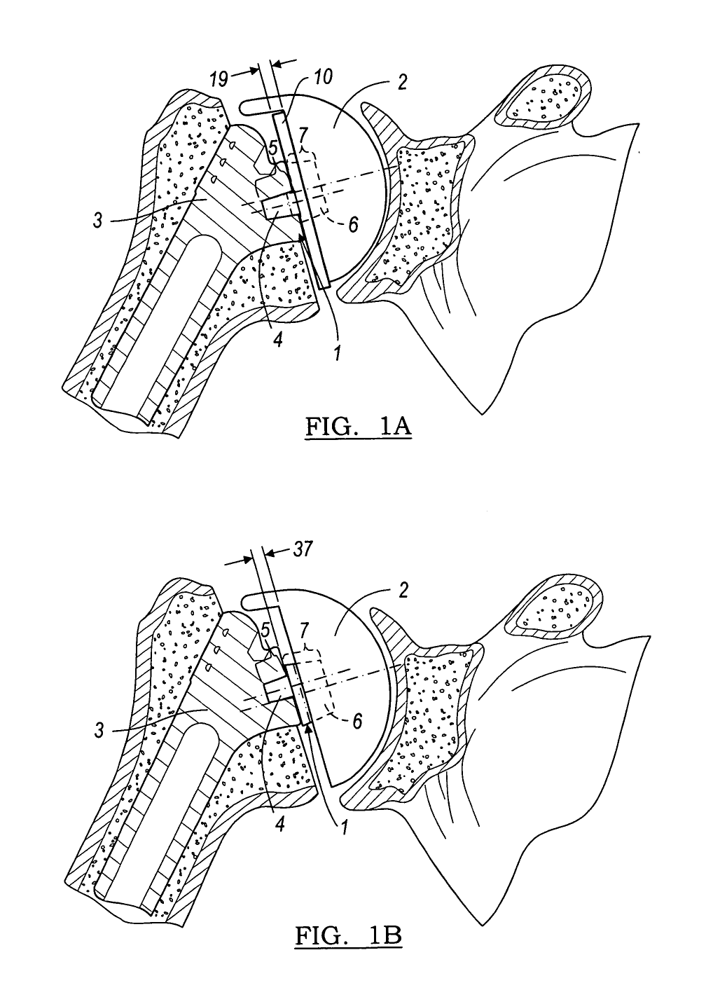

FIGS. 1A-1D reference a joint prosthesis system having an adaptor prosthesis 1 coupled to an articulation head 2 and a stem 3. Adaptor prosthesis 1 has a coupling mechanism 4 that is formed of a male coupling taper 5. This coupling taper 5 is inserted into a receptor within articulation head 2. Adaptor prosthesis 1 further includes a main body 6 that also forms a male coupling taper 7 for insertion into stem 3. It should be noted that the connection mechanism between adaptor prosthesis 1 and articulation head 2; and between adaptor prosthesis 1 and stem 3 may encompass many different spatial configurations. For example, the coupling mechanism 4 and main body 6 could both form female tapers or a combination of at least one male taper and/or at least one female taper.

The head 2 can have an extended articulating portion or surface 11. The extending articulating portion 11 can be radially located at any position about the fixation member 3. When located at a superior position with respect to the fixation member 3, the extending articulating member 2 allows the head to articulate with acromion in cases where the rotator cuff is deficient or absent.

As shown in FIG. 1D, the centerline of the coupling mechanism 4 of the head 2 can be offset from a centerline that defines the articulating surface or a centerline that is defined by the adaptor prosthesis 1. When used in conjunction with an adaptor prosthesis 1 having an offset coupling mechanism 4, the location of the extended articulating portion with respect to the stem 3 can vary greatly.

The use of an adaptor 1 having an increased height allows for the use of an extended articulating head 2 having an articulating surface with a reduced radius. In this regard, the increased height allows for the extension 11 will not interfere with the tuberosity (not shown) associated with the humeral head complex. Further, the adaptivity of the offset head 2, along with the offset associated with the adaptor 1, allows for increased flexibility for the physician to create a maximum range of radial offset while allowing the tensioning of the muscle complexes needed to prevent the aforementioned dislocations.

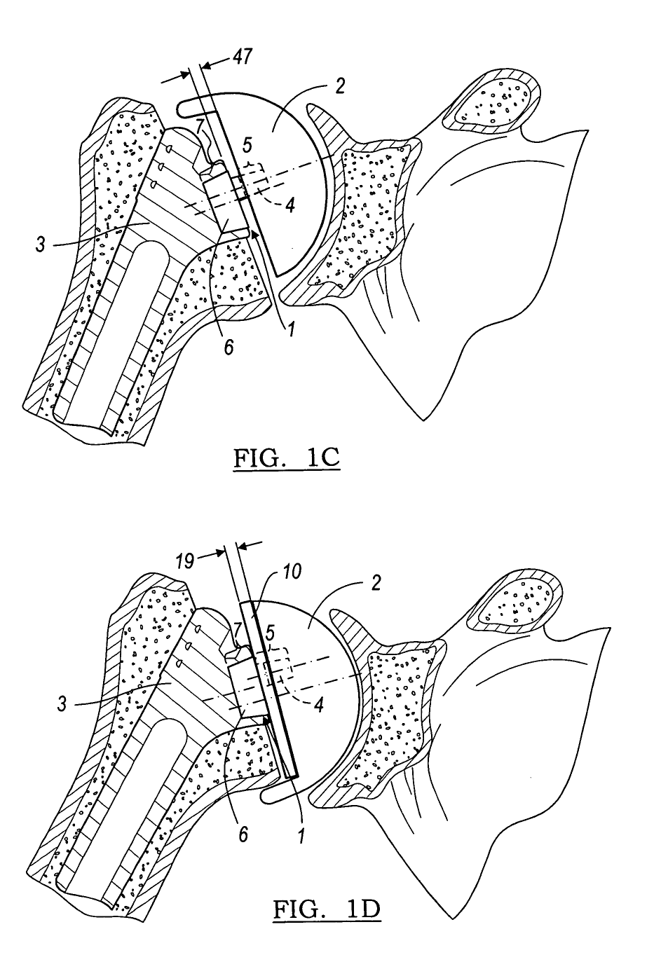

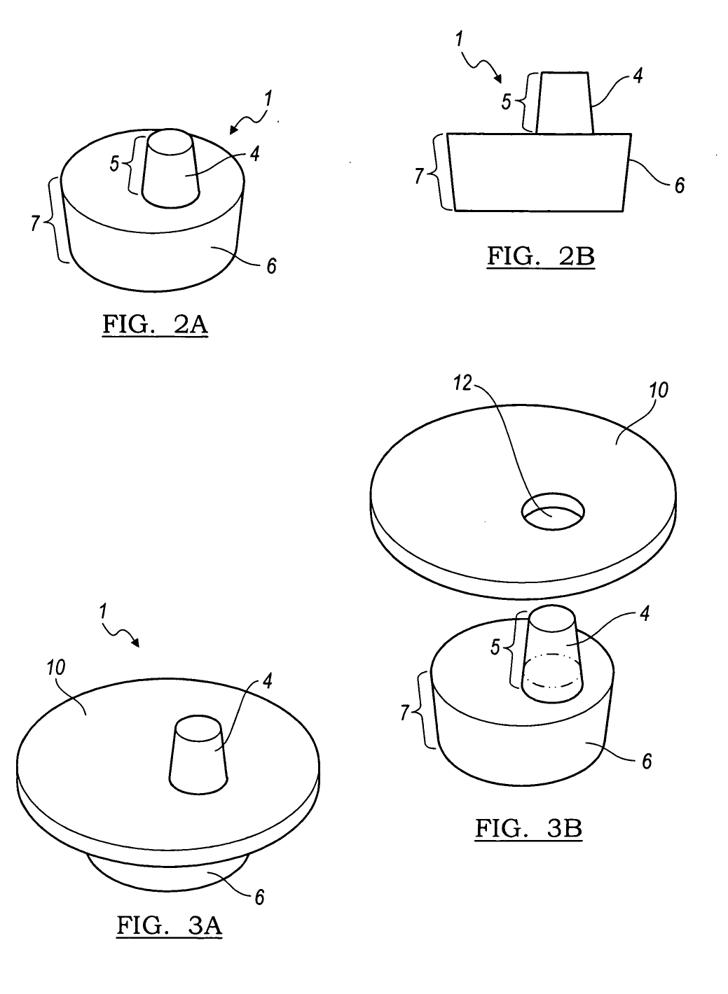

FIGS. 2A-2B disclose a perspective and side view of adaptor prosthesis 1 from the joint prosthesis system disclosed in FIGS. 1A-1C. FIG. 3A shows adaptor prosthesis 1 incorporated with a spacer 10. With reference to FIG. 1A, spacer 10 is used to fill the offset of the articulation head 2 from the oppositely located stem 3. Spacer 10 is used to constrain motion between articulation head 2 and stem 3. FIG. 3B further discloses an exploded view of adaptor prosthesis 1 and spacer 10, having an aperture 12. FIG. 3C illustrates male taper 7 of main body 6 and the corresponding male taper 5 of coupling mechanism 4 and aperture 18 of spacer 10. Additionally shown is an optional anti-rotation peg which is configured to interface with an aperture defined in the spacing plate. Coupling mechanism 4 is inserted through aperture 12 of spacer 10, which is configured to receive male taper 5 via aperture taper 18. Aperture taper 18 of aperture 12 is manufactured to fit snugly to male taper 5 of coupling mechanism 4 at the base of main body 6. Spacer 10 is configured to fill the offset of the articulation head 2 from stem 3 by spacer height 19. A person of ordinary skill in the art would appreciate that there are various possible male/female connector combinations consistent with the exemplary disclosure.

FIGS. 3D-3E illustrate the combination of adaptor prosthesis I and spacer 10, where aperture taper 18 of spacer 10 is engaged around male taper 5 of coupling mechanism 4. Spacer 10 is located at the base of main body 6. It is envisioned the spacer 10 can be secured to adaptor prosthesis 1 by interaction between the tapers. Additionally, spacer 10 could be fixed to the base of coupling mechanism 4 or main body 6 using a threaded fastener.

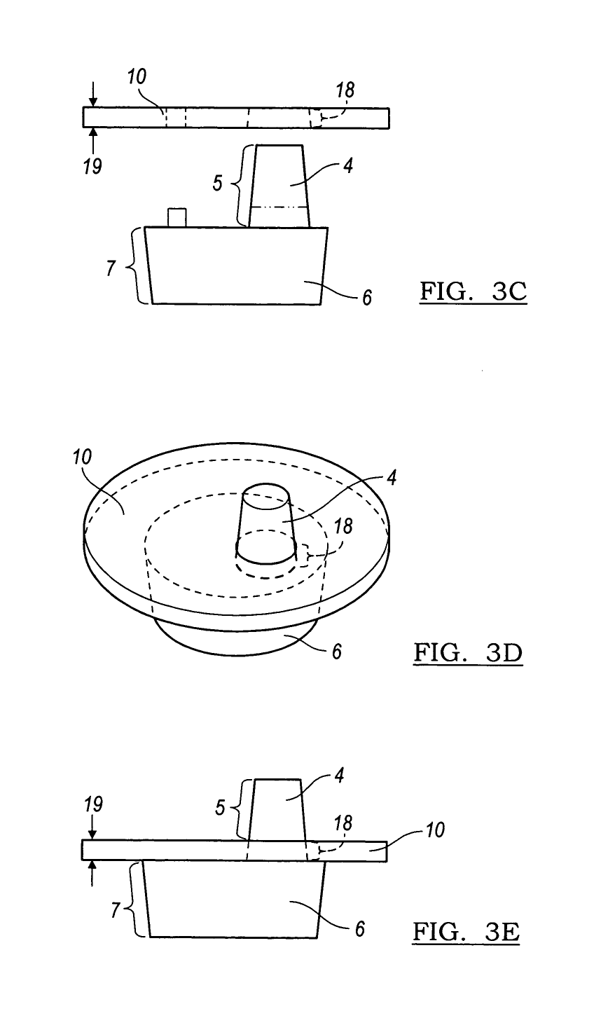

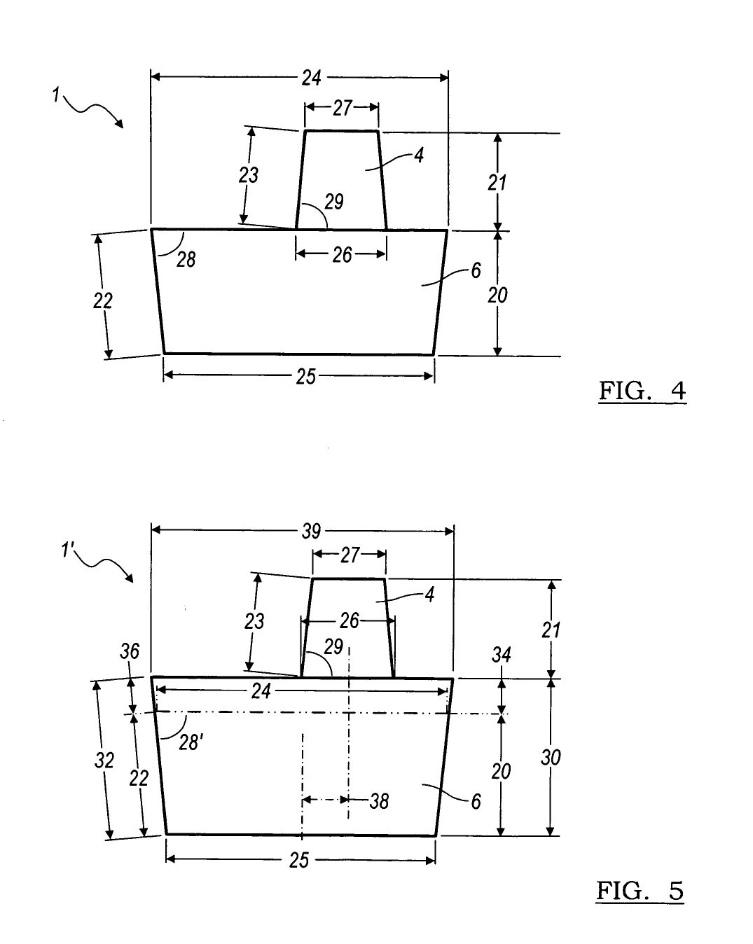

FIGS. 4-7 depict several exemplary illustrations of the present adaptor prosthesis kit of the present disclosure. FIG. 4 illustrates adaptor prosthesis 1 as configured with a main body height 20, a coupling mechanism height 21, a main body length 22, and a coupling mechanism length 23. In accordance with the disclosure, main body height 20, coupling mechanism height 21, main body length 22, or coupling mechanism length 23 may be increased or decreased in height or length to obtain varying offset distances between the base of an articulating prosthetic head 2 and the base of a fixation component 3. FIG. 4 also illustrates main body major width 24, main body minor width 25, coupling mechanism major width 26, coupling mechanism minor width 27, a main body angle 28, and a coupling mechanism angle 29. Likewise, the present disclosure contemplates changes in vertical offset between the base of an articulating prosthetic head and a base fixation component by varying the lengths, widths, and angles of main body 6. To this end, the major width 24, main body minor width 25, coupling mechanism major width 26, coupling mechanism minor width 27, main body angle 28, or coupling mechanism angle 29. While FIG. 4 illustrates an exemplary adaptor prosthesis 1 configuration, it is envisioned that vertically offsetting the base of an articulating prosthetic head from the base of a fixation component may be accomplished with various shapes, sizes, and configurations. These features of the present disclosure will become apparent from the description of FIGS. 5-8.

With continued reference to FIG. 4, the height of adaptor prosthesis 1 is represented by either main body height 20 or coupling mechanism height 21. Main body height 20 represents the vertical distance from the base of main body 6 to the top of main body 6. Likewise, coupling mechanism height 21 is the vertical distance from the base of coupling mechanism 4 to the top of coupling mechanism 4. Similarly, the length of adaptor prosthesis 1 may be represented by main body length 22 or coupling mechanism length 23. Main body length 22 varies from main body height 20 in that main body length 22 is the hypotenuse of the triangle formed by the complementary intersection of main body height 20 and one half the difference between main body major width 24 and main body minor width 25. Similarly, coupling mechanism length 23 varies from coupling mechanism height 21 in that coupling mechanism length 23 is the hypotenuse of the triangle formed by the complementary intersection of coupling mechanism height 21 and one half the difference between coupling mechanism major width 26 and coupling mechanism minor width 27. Corresponding changes in main body major width 24 or main body minor width 25 will change main body angle 28; and vice versa. Changes in coupling mechanism major width 26 or coupling mechanism minor width 27 will effectively change coupling mechanism angle 29; and vice versa.

The adaptor prosthesis 1′ in FIG. 5 illustrates an extended main body 6 as represented by either the second main body height 30 or the second main body length 32. The difference in height between main body height 20 in FIG. 4 and second main body height 30 in FIG. 5 is the main body height offset 34. Similarly, the difference in length between main body length 22 in FIG. 4 and second main body length 32 in FIG. 5 is the main body length offset 36. Creating an offset distance using main body height offset 34, main body length offset 36, or a combination of the two, is in accord with the present disclosure. Corresponding FIG. 1B depicts the extended vertical offset 34 of main body 6 between articulation head 2 and stem 3. Unless the ratio between main body major width 24 and main body minor width 25 changes, main body angle 28 in FIG. 4 is equivalent to main body angle 28′ in FIG. 5.

With continued reference to FIG. 5, radial offset 38 is the distance between the center of coupling mechanism 4 and main body 6. Optionally, this offset can be between 0 and about 10 mm. Offsetting the center of coupling mechanism 4 from main body 6 by radial offset 38 facilitates angular or radial offset of the articulation head relative to the fixation component. These offsets provide a larger range of configurations which provide a better opportunity to more adequately reproduce natural movement.

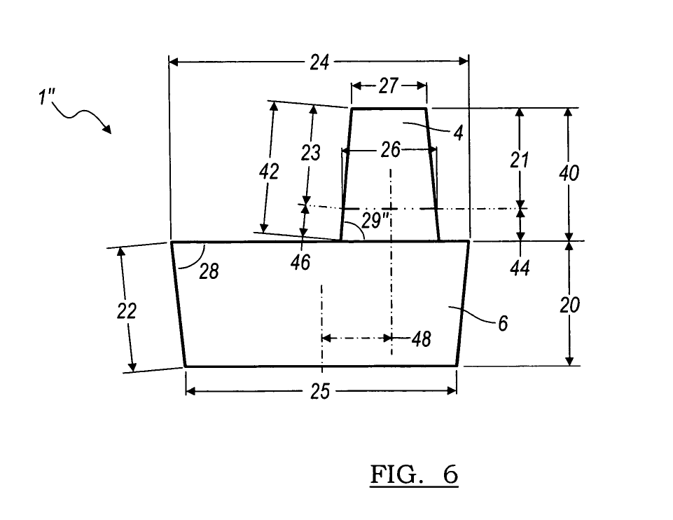

Adaptor prosthesis 1″ in FIG. 6 discloses the incorporation of an extended coupling mechanism 4. FIG. 6 illustrates a second coupling mechanism height 40 and a second coupling mechanism length 42. The difference in height between coupling mechanism height 21 in FIG. 4 and second coupling mechanism height 40 in FIG. 6 is the coupling mechanism . height offset 44. Similarly, the difference in length between coupling mechanism length 23 in FIG. 4 and second coupling mechanism length 42 in FIG. 6 is the coupling mechanism length offset 46. Creating an offset distance using coupling mechanism height offset 44, coupling mechanism length offset 46, or a combination of the two, is in accord with the present disclosure. Corresponding FIG. 1C depicts the extended vertical offset 47 of main body 6 between articulation head 2 and stem 3. Unless the ratio between coupling mechanism major width 26 and coupling mechanism minor width 27 changes, coupling mechanism angle 29 in FIG. 4 is equivalent to coupling mechanism angle 29″ in FIG. 6.

Furthermore, a second radial offset 48 is disclosed as the distance between the center of coupling mechanism 4 and main body 6. Offsetting the center of coupling mechanism 4 from main body 6 by a radial offset 48 further facilitates angular or radial offset of the articulation head relative to the fixation component. These offsets provide a larger range of configurations which provide a better opportunity to achieve the natural range of motion after total shoulder or hip prosthesis reconstruction.

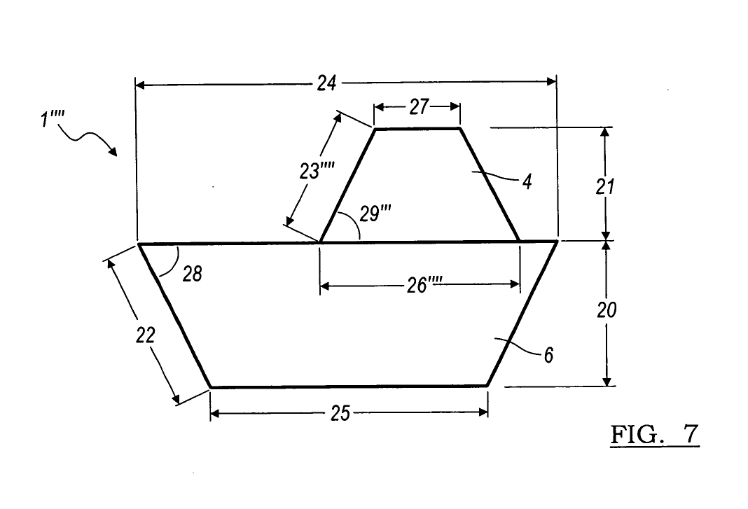

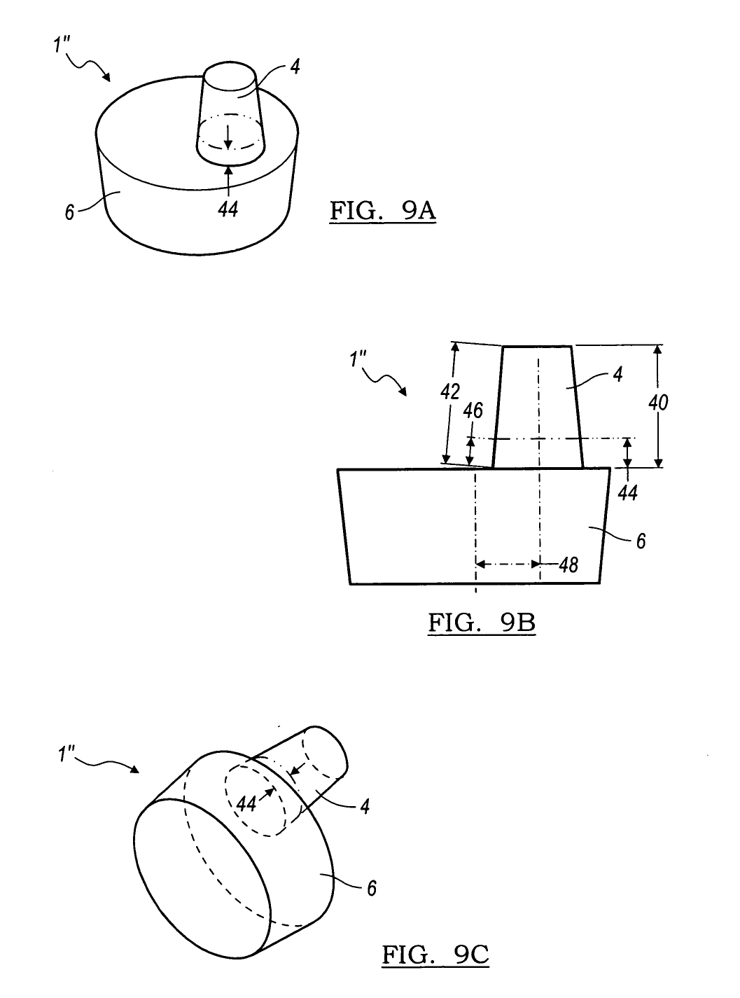

FIG. 7 illustrates adaptor prosthesis 1″″ having an alternate coupling mechanism 4 configuration. Relative to coupling mechanism major width 26 in FIG. 4, coupling mechanism major width 26″″ in FIG. 7 is elongated. In turn, when coupling mechanism minor width 27 is held constant, coupling mechanism length 23″″ increases in length and coupling mechanism angle 29″″ decreases. Alternatively if the ratio between coupling mechanism major width 26″″ and coupling mechanism minor width 27 decreases, coupling mechanism length 23″″ would be shorter than coupling mechanism length 23 in FIG. 4 and coupling mechanism angle 29″″ in FIG. 9 would be greater than coupling mechanism angle 29 in FIG. 4. Varying one or more of the aforementioned widths, lengths, heights, or angles is also within the scope of the present disclosure.

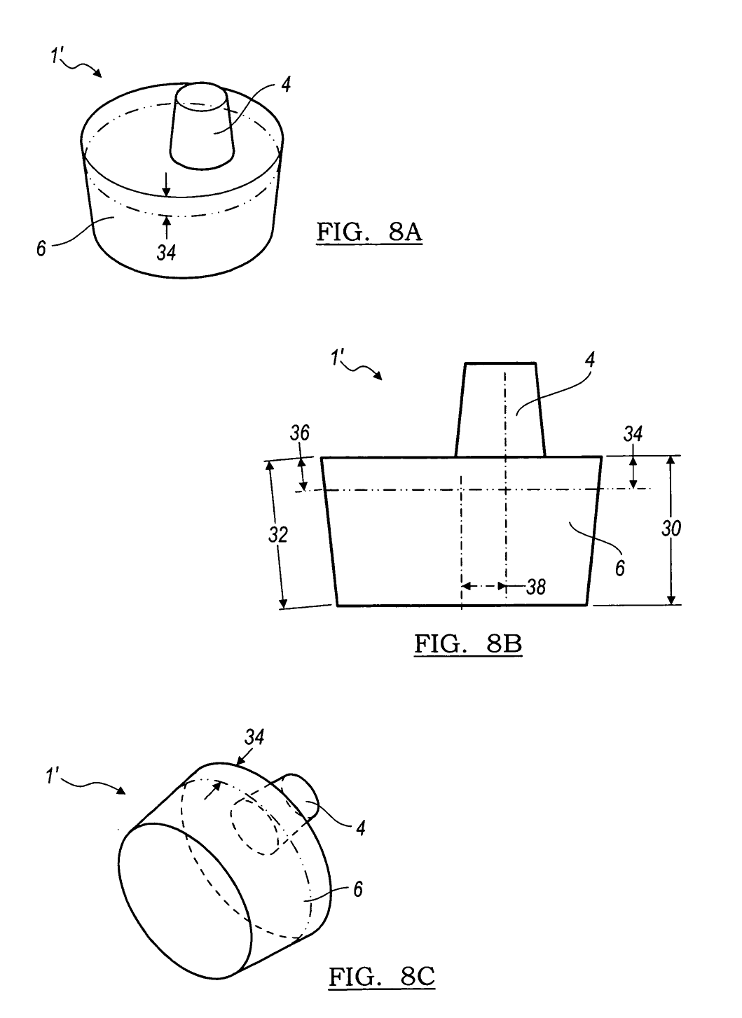

FIG. 7 further illustrate adaptor prosthesis 1′ disclosed in FIG. 5. FIGS. 8A-8C show three different perspectives of main body height offset 34 in conjunction with main body 6, as previously described, in accordance with the present disclosure. Similarly, FIGS. 9A-9C further illustrate adaptor prosthesis 1″ of FIG. 5. FIGS. 9A-9C show different perspectives of coupling mechanism height offset 44. Additionally, FIG. 8B and FIG. 9B illustrate radial offset 38 and second radial offset 48, respectively.

In reference to all of the above-described embodiments, various tapered surfaces have been referenced with the adaptor prosthesis, articulating prosthetic head, and fixation component. As presently disclosed, these tapered surfaces are configured as morse-type tapers which provide a self locking interface. While Morse-type tapers are presently preferred, one skilled in the art will readily recognize that other means may be incorporated for providing a locking interface between the various components of the kit of adaptors. In this regard, one or more interfaces may be interlocked with the use of an additional fastener to insure locking engagement therebetween. Alternately, interfacing surfaces can have coupling surfaces which intersect each other so as to facilitate locking.

While the present disclosure has been described in several exemplary forms, it is to be understood there are numerous applications and implementations for the present disclosure. Accordingly, modifications and changes to the exemplary models set out in the disclosure may be made without departing from the sprit and scope of this disclosure.

What is claimed is:

1. A kit of prosthetic components comprising:
a first adaptor having a first end and a second end, said first end defining a first coupling member having a first coupling surface, said first coupling member extends along a first axis and said second end defines a second coupling member having a second coupling surface, said second coupling member extends along a second axis, wherein said first coupling member has a first height that extends from said first end to said second coupling member; and
a second adaptor having a first end and a second end, said first end defining a third coupling member having a third coupling surface, said first coupling member extends along a third axis and said second end defines a fourth coupling member having a fourth coupling surface, said fourth coupling member extends along a fourth axis, wherein said second adaptor has a second height that extends from said first end to said fourth coupling member; wherein said first height is different from said second height.

2. The kit according to claim 1, wherein said first axis is colinear with said second axis.

3. The kit according to claim 1, wherein said first coupling member and said third coupling member form male locking tapers.

4. The kit according to claim 1, wherein said second coupling member and said fourth coupling member form male locking tapers.

5. The kit according to claim 1, wherein said second coupling member has a third height and the fourth coupling member has a fourth height which is greater than said third height and wherein the first height is parallel with the first axis.

6. The kit according to claim 1, further comprising a stem, a head, and a glenoid.

7. The kit according to claim 6, wherein the head defines an external perimeter, said external perimeter defining a central head axis, said head defining a female coupling taper, said female coupling taper defining a head coupling axis.

8. The kit according to claim 7, wherein the head coupling axis is offset from the central head axis.

9. The kit according to claim 7, wherein the head comprises an extended articulating surface.

10. The kit according to claim 9, wherein the central head axis is configured to be disposed between the head coupling axis and the extended articulating surface.

11. A kit of prosthetic components comprising:
a first adaptor having a first end and a second end, said first end defining a first coupling member that extends along a first axis and said second end defines a second coupling member that extends along a second axis, wherein said first coupling member has a first length that extends from said first end to said second coupling member;
a second adaptor having a first end and a second end, said first end defining a third coupling member that extends along a third axis and said second end defines a fourth coupling member that extends along a fourth axis, wherein said second adaptor has a second height that extends from said first end to said fourth coupling member; and wherein said first length is different from said second length.

12. The kit according to claim 11, wherein said first adaptor and said second adaptor are generally cylindrical.

13. The kit according to claim 11, wherein said second coupling member and said fourth coupling member are of equal length.

14. The kit according to claim 11, wherein said first adeapter is configured to be coupled to a bone fixation member and wherein the second coupling member is configured to be coupled to an articulating head.

15. The kit according to claim 11, further comprising a head configured to mate with one of the first or second adaptor.

16. The kit according to claim 15, wherein the head defines an external perimeter, said external perimeter defining a central head axis, said head defining a female coupling taper, said female coupling taper defining a head coupling axis which is offset from the central head axis.

17. The kit according to claim 11, wherein said first coupling member and said second coupling member form male Morse tapers.

18. The kit according to claim 17, wherein said third and fourth coupling members form a Morse taper.

19. The kit according to claim 11, further comprising a stem, a head, a spacer, and a glenoid, each of which are configured to be coupled to the first adaptor.

20. The kit according to claim 19, wherein the head comprises an extended articulating surface and wherein the central head axis is disposed between the head coupling axis and the extended articulating surface.

21. A kit of prosthetic components, comprising:
a first adaptor having a first main body having a first coupling surface and a first coupling mechanism having a second coupling surface, wherein said first adaptor is adapted to be coupled to a head having an articulating surface and adapted to be coupled to a fixation component, the distance between said articulating surface and fixation component is defined by a first distance between said first and second coupling surfaces; and
a second adaptor having a second main body having a third coupling surface and a second coupling mechanism having a fourth coupling surface, wherein the second adaptor is adapted to be coupled to the head and is adapted to be coupled to the fixation component, the distance between said articulating surface and said fixation component is defined by a second distance between said third and fourth coupling surfaces and wherein the first distance is different than the second distance.

22. The kit according to claim 21 wherein the first main body defines a first axis and the first adaptor defines a second axis.

23. The kit according to claim 22, wherein the first axis of said first main body is radially offset from the second axis of said first adaptor.

24. The kit according to claim 21, wherein a first axis of said second main body is radially offset from a second axis of said second coupling mechanism.

25. The kit according to claim 24, wherein the offset between the first axis and the second axis is between 0 and about 10 millimeters.

26. The kit according to claim 21, wherein said fixation component is a humeral stem.

27. The kit according to claim 21, further comprising a prosthetic glenoid.

28. The kit according to claim 21, further comprising a stem and an articulating head.

29. A method for reducing likelihood of dislocations in a shoulder joint comprising:
coupling a humeral fixation member to a humerus;
determining a proper size for an articulating head to be coupled to the fixation member;
determining the proper medial displacement of the head from the fixation member;
determining a proper superior displacement of the head with respect to the humeral fixation; and
disposing an adaptor between the head and the fixation member to provide the medial and superior displacement.

30. The method according to claim 29, further comprising providing an extended articulating bearing surface on the head at a location superior to the adaptor.

31. The method according to claim 29, wherein determining the proper superior displacement is determining the proper superior displacement sufficient to provide a predetermined amount of tension on a deltoid muscle.
